(19) United States
(12) Patent Application Publication
(10) Pub. No.: US 2023/0406899 A1
(43) Pub. Date: Dec. 21, 2023

(54) PRODUCTS AND METHODS FOR THE DIAGNOSIS AND DIFFERENTIATION OF HEPARIN-INDUCED THROMBOCYTOPENIA FROM VACCINE-INDUCED IMMUNE THROMBOTIC THROMBOCYTOPENIA AND NON-HEPARIN-INDUCED THROMBOCYTOPENIA

(71) Applicant: McMaster University, Hamilton (CA)

(72) Inventors: Ishac Nazy, Hannon (CA); Donald Arnold, Hamilton (CA); John Kelton, Hamilton (CA); Angela Huynh, Hamilton (CA); Mercy Charity Daka, Mississauga (CA)

(21) Appl. No.: 18/211,970

(22) Filed: Jun. 20, 2023

Related U.S. Application Data
(60) Provisional application No. 63/353,308, filed on Jun. 17, 2022.

(51) Int. Cl. C07K 14/52; G01N 33/68; G01N 33/564

(52) U.S. Cl. CPC C07K 14/522; G01N 33/6863; G01N 2333/522; G01N 2800/226; G01N 33/564

(57) ABSTRACT

Described are mutant Platelet Factor 4 (PF4) proteins that exhibit different binding affinities to vaccine induced immune thrombotic thrombocytopenia (VITT) antibodies or non-heparin-induced thrombocytopenia (non-HIT) antibodies relative to heparin-induced thrombocytopenia (HIT) antibodies. Also provided herein are methods for differentiating between VITT, HIT, and/or non-HIT in subjects suspected of having VITT, HIT, or non-HIT.

### RELATED APPLICATIONS

This application claims the benefit of U.S. provisional patent application No. 63/353,308 filed on Jun. 17, 2022, herein incorporated by reference.

### INCORPORATION OF SEQUENCE LISTING

A computer readable form of the Sequence Listing “3244-P65075US02_SequenceListing” (2100 bytes), submitted via Patent Center and created on Jun. 19, 2023, is herein incorporated by reference.

### FIELD

The present disclosure relates to mutant platelet factor 4 (PF4) proteins and more specifically to mutant PF4 proteins and methods for the differentiation of heparin-induced thrombocytopenia (HIT) from vaccine-induced immune thrombotic thrombocytopenia (VITT) and heparin-induced thrombocytopenia from non-heparin-induced thrombocytopenia (Non-HIT).

### BACKGROUND

Vaccine-induced immune thrombotic thrombocytopenia (VITT) is a rare, but serious adverse effect of adenoviral vector vaccines against SARS-CoV-2 virus. The clinical picture of VITT is moderate to severe thrombocytopenia plus arterial and/or venous thrombi, often occurring in unusual locations¹⁻³. These findings resemble the immunological drug-reaction called heparin-induced thrombocytopenia (HIT), which presents clinically as thrombocytopenia and thrombosis in patients previously exposed to heparin⁴⁻⁶. VITT mostly resembles the rare incidents of spontaneous HIT, which occurs in the absence of heparin^{8,9}.

HIT is caused by immunoglobulin G (IgG) antibodies that bind to neoepitopes on platelet factor 4 (PF4), a 70 amino acid cationic protein contained within platelets¹⁰⁻¹². The neoepitopes become exposed after heparin, a large anionic polysaccharide, clusters PF4 tetramers. The IgG-specific antibodies bind to PF4/heparin and form immune complexes, which activate platelets through their Fc γRIIa receptors causing intense platelet activation and the release of procoagulant rich microparticles¹³. Other cells, including monocytes, are also activated by these immune complexes, which amplifies the hypercoagulable state in HIT patients¹⁴. It has been postulated that VITT has a similar pathophysiology to HIT, and several investigators have demonstrated the presence of high levels of anti-PF4 antibodies in VITT sera¹⁻³. However, VITT is a unique syndrome since it occurs without heparin exposure, and the pattern of platelet reactivity does not demonstrate typical heparin-dependence, as seen with HIT. Therefore, there is a need for tests to differentiate between HIT and VITT as well as HIT and non-HIT in order to optimize treatment for patients.

### SUMMARY

The present disclosure describes the characteristics of VITT antibodies that develop in response to COVID-19 adenoviral vector vaccination. The present inventors identified mutant Platelet Factor 4 (PF4) proteins that exhibit different binding affinities to vaccine induced immune thrombotic thrombocytopenia (VITT) antibodies relative to heparin-induced thrombocytopenia (HIT) antibodies as well as mutant PF4 proteins that exhibit different binding affinities to HIT than to non-HIT antibodies. The inventors further identified methods for the detection of VITT antibodies vs non-HIT antibodies vs HIT antibodies and/or for classifying a subject as having VITT, non-HIT, or HIT.

Accordingly, provided herein is a mutant Platelet Factor 4 (PF4) protein comprising a mutation to at least one amino acid selected from positions R20, H23, T25, E28, K46, R49, K50, K62, K65, or K66 of the amino acid sequence of SEQ ID NO:1.

In an embodiment, the mutation comprises substitution of one or more mutations selected from R20A, H23A, T25A, E28A, K46A, R49A, K50A, K62A, K65A, or K66A to the amino acid sequence of SEQ ID NO:1.

In another embodiment, the mutant PF4 protein:
(a) selectively binds a heparin-induced thrombocytopenia (HIT) antibody compared to a vaccine-induced immune thrombotic thrombocytopenia (VITT) antibody (PF4-H⁺V⁻);
(b) selectively binds the HIT antibody compared to a non-HIT antibody (PF4-H⁺NH⁻); and/or
(c) selectively binds the HIT antibody compared to both the VITT antibody and the non-HIT antibody (PF4-H⁺V⁻NH⁻).

In an embodiment, the PF4-H⁺V⁻ comprises the substitution R20A, H23A, T25A, E28A, K46A, R49A, K50A, K62A, K65A, or K66A to the amino acid sequence of SEQ ID NO:1. In another embodiment, the PF4-H⁺V⁻ comprises the substitution H23A, E28A, K50A, K62A, or K66A to the amino acid sequence of SEQ ID NO:1. In yet another embodiment, the PF4-H⁺V⁻ comprises the substitution H23A, E28A, K50A, or K66A to the amino acid sequence of SEQ ID NO:1.

In an embodiment, the PF4-H⁺NH⁻ comprises the substitution H23A, T25A, R49A, K65A, or K66A to the amino acid sequence of SEQ ID NO:1. In another embodiment, the PF4-H⁺NH⁻ comprises the substitution H23A, T25A, or K65A to the amino acid sequence of SEQ ID NO:1.

In an another the PF4-H⁺V⁻NH⁻ comprises the substitution H23A, T25A R49A, K65A, or K66A to the amino acid sequence of SEQ ID NO:1.

Also provided herein is a method of differentiating between VITT, HIT, and non-HIT in a sample from a patient suspected of having VITT, HIT or non-HIT comprising:
a) contacting the sample with at least one of the mutant PF4 protein provided herein and measuring binding of the at least one mutant PF4 protein with the sample;
b) contacting the sample with the PF4 protein comprising the amino acid sequence of SEQ ID NO:1 and measuring the binding of the PF4 protein with the sample;
c) determining a ratio of the binding in a) to the binding in b); and
d) determining the level of similarity of the ratio in c) to one or more control profiles, wherein (i) a high level of similarity to a HIT specific control profile or a low level of similarity to a VITT specific control profile and/or a non-HIT specific control profile is indicative of HIT; (ii) a high level of similarity to the VITT specific control profile or a low level of similarity to the HIT specific control profile and/or the non-HIT specific control profile is indicative of VITT; and/or (iii) a high level of similarity to the non-HIT specific control profile or a low level of similarity to the HIT specific control profile and/or the VITT specific control profile is indicative of non-HIT.

In an embodiment, the method first comprises the step of immobilizing the mutant PF4 protein in a) and/or the PF4 protein in b) on a support, optionally a biosensor tip or microplate.

In another embodiment, the mutant PF4 protein in a) and/or the PF4 protein in b) is biotinylated.

In yet another embodiment, the mutant PF4 protein in a) and/or the PF4 protein in b) is immobilized alone or complexed with unfractionated heparin.

In an embodiment, the method comprises enzyme-linked immunosorbent assay (ELISA), enzyme immunoassay (ETA), or bio-layer interferometry (BLI).

In another embodiment, the measuring in step a) and/or b) comprises measuring the detection of a secondary antibody tagged with a detectable label against the HIT antibody, the VITT antibody, and/or the non-HIT antibody bound to the mutant PF4 protein or the PF4 protein comprising the amino acid sequence of SEQ ID NO: 1.

In yet another embodiment, the measuring in step a) and/or step b) comprises measuring a colorimetric shift in optical interference pattern induced by binding of the HIT antibody, the VITT antibody, and/or the non-HIT antibody to the mutant PF4 protein or the PF4 protein comprising the amino acid sequence of SEQ ID NO:1.

Also provided herein is a method of differentiating between VITT and HIT in a sample from a patient suspected of having VITT or HIT comprising:
I) combining the sample with platelets labeled with serotonin;
II) incubating the combined sample and platelets with no unfractionated heparin and at least two concentrations of the unfractionated heparin, and measuring serotonin release to the no unfractionated heparin and to the at least two concentrations of the unfractionated heparin;
III) incubating the combined sample and platelets with no mutant PF4 protein and at least two concentrations of the mutant PF4 protein herein disclosed, and measuring the serotonin release to the no mutant PF4 protein and to the at least two concentrations of the mutant PF4 protein;
IV) measuring a dose response of the sample in II) and in III); and
V) determining the level of similarity of the dose response in IV) to one or more control profiles, wherein (i) a high level of similarity to a HIT specific control profile or a low level of similarity to a VITT specific control profile is indicative of HIT; and/or (ii) a high level of similarity to the VITT specific control profile or a low level of similarity to the HIT specific control profile is indicative of VITT.

In an embodiment, the platelets in I) are preincubated with FcγRIIa-blocking monoclonal antibody (IV.3).

In another embodiment, the HIT specific control profile comprises a non-null dose response to both i) the unfractionated heparin, and ii) the mutant PF4 protein; and/or wherein the VITT specific control profile comprises a null dose response to i) the unfractionated heparin, and the non-null dose response to ii) the mutant PF4 protein.

In yet another embodiment, the sample is a blood sample, optionally, a serum or plasma sample.

These and other features and advantages of the present disclosure will become apparent from the following detailed description taken together with the accompanying drawings. It should be understood, however, that the detailed description and the specific examples, while indicating embodiments of the present disclosure, are given by way of illustration only and the scope of the claims should not be limited by these embodiments, but should be given the broadest interpretation consistent with the description as a whole. Various changes and modifications within the spirit and scope of the disclosure will become apparent to those of skill in the art from this detailed description.

### DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1A-B shows platelet activation (% serotonin release) of sera from VITT patients (grey lines) and a representative HIT patient (black line) the standard SRA with added heparin²⁵ (FIG. 1A) and PF4-enhanced SRA^{26,27} with added PF4 (FIG. 1B) in an exemplary embodiment of the disclosure. The horizontal dashed bar represents the cutoff used for the SRA where a ¹⁴C-serotonin release≥20% is considered positive. Results demonstrate that in the standard SRA, the HIT serum has a dose-dependent response to both heparin and PF4 while the VITT sera were only able to activate platelets with the addition of PF4. All platelet activation was inhibited by the addition of IV.3 monoclonal antibody that binds the FcγRIIa receptors on platelets.

FIG. 2A-D shows epitope mapping of amino acids on PF4 that are critical for binding antibodies from VITT and HIT sera, using alanine scanning mutagenesis in an exemplary embodiment of the disclosure. VITT sera (n=5) showed that the binding region (darkened region in FIG. 2A) aligns within the heparin binding region on PF4 (darkened region in FIG. 2B). When testing HIT sera (n=10), one main binding region was identified for all sera as shown in the darkened region in FIG. 2C as well as an additional binding region in 6/10 of the HIT sera (expanded darkened region in FIG. 2D as compared to FIG. 2C), which aligns within the heparin binding region on PF4. Images are modified from PDB 1RHP.

FIG. 3A-D shows binding responses and dissociation rates (k_{off}) of sera from VITT patients (n=5), HIT patients (n=10), and healthy controls (n=10) binding to PF4 and PF4/heparin, using BLI in an exemplary embodiment of the disclosure. FIG. 3A-B show that VITT sera have higher binding responses to both PF4 (FIG. 3A) and) PF4/heparin (FIG. 3B) than either HIT sera or sera from healthy controls. FIG. 3C-D shows that dissociation rates were variable in VITT and HIT patients to PF4 (FIG. 3C) and PF4/heparin (FIG. 3D); however, they were not significantly different. Values are shown as a mean binding response (nm)±SD and mean dissociation rates k_{off} (s−1)±SD. ***p<0.001, ****p<0.0005.

FIG. 4 shows differences in binding of VITT and HIT patient sera with PF4 mutants in an exemplary embodiment of the disclosure. Eight separate PF4 mutants were tested to identify a new diagnostic ELISA that can separate VITT from HIT sera. Only mutants R22A, H23A and K46A were shown to have a statistically significant (R22A and K64A: *p<0.05, H23A: **p<0.01) difference between VITT and HIT sera, with the VITT sera showing a higher binding sensitivity to these amino acid changes. Results are shown as a percentage of binding when compared to the wild-type PF4 protein comprising the amino acid sequence of SEQ ID NO:1 (Table 1).

FIG. 5 shows a schematic of a proposed mechanism of VITT antibodies binding to and clustering PF4 tetramers, independent of heparin, and forming platelet-activating immune complexes in an exemplary embodiment of the disclosure.

FIG. 6 shows updated data on the selected PF4 mutants that differentiate VITT from HIT antibodies in an exemplary embodiment of the disclosure. The 10 PF4 mutants selected, are those of which the corresponding amino acids are important either for VITT antibody binding in the first alanine scanning mutagenesis screen or the heparin binding on PF4. Each point represents the binding ability of VITT (n=9) and HIT (n=22) serum samples to 10 PF4 mutants (R20A, H23A, T25A, E28A, K46A, R49A, K50A, K62A, K65A, K66A) relative to wild-type PF4. Data reported as mean PF4 mutant binding relative to wild-type PF4 and error bars correspond to the standard deviation. Black closed circles represent the binding of VITT patient sera and the black open circles represent HIT patient sera. Student's t-test was performed to determine the significant differences between the two antibody groups. **p<0.01, ***p<0.001, ****p<0.0001.

FIG. 7 shows updated data on the selected PF4 mutants that differentiate HIT from non-HIT antibodies in an exemplary embodiment of the disclosure. The 10 PF4 mutants selected, are those of which the corresponding amino acids are important either for VITT antibody binding in the first alanine scanning mutagenesis screen or the heparin binding on PF4. Each point represents the binding ability of HIT (n=22) and non-HIT (n=12) serum samples to 10 PF4 mutants (R20A, H23A, T25A, E28A, K46A, R49A, K50A, K62A, K65A, K66A) relative to wild-type PF4. Data reported as mean PF4 mutant binding relative to wild-type PF4 and error bars correspond to the standard deviation. Black closed circles represent the binding of HIT patient sera and the black open circles represent non-HIT patient sera. Student's t-test was performed to determine the significant differences between the two antibody groups. *p<0.05, **p<0.01, ****p<0.0001.

Further aspects and features of the example embodiments described herein will appear from the following description taken together with the accompanying drawings.

### DETAILED DESCRIPTION

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein, except combinations where at least some of such features and/or steps are mutually exclusive. Further, any feature indicated as being preferred or advantageous may be combined with any other feature or features indicated as being preferred or advantageous.

### I. Definitions

As used herein, the following terms may have meanings ascribed to them below, unless specified otherwise. Unless otherwise indicated, the definitions and embodiments described in this and other sections are intended to be applicable to all embodiments and aspects of the present disclosure herein described for which they are suitable as would be understood by a person skilled in the art. In the case of conflict, the present specification, including definitions, will control. In addition, the materials, methods, and examples are illustrative only and not intended to be limiting.

In understanding the scope of the present disclosure, the term “comprising” and its derivatives, as used herein, are intended to be open ended terms that specify the presence of the stated features, elements, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 50%, 5-50%, or 10-40%, 10-20%, 10%-15%, preferably 5-10%, most preferably about 5% of the number to which reference is being made.

As used in this disclosure, the singular forms “a”, “an” and “the” include plural references unless the content clearly dictates otherwise. Thus, for example, a composition containing “a compound” includes a mixture of two or more compounds.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present. Other elements may optionally be present other than the elements specifically identified by the “and/or” clause, whether related or unrelated to those elements specifically identified.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of’ or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

It should also be understood that, in certain methods described herein that include more than one step or act, the order of the steps or acts of the method is not necessarily limited to the order in which the steps or acts of the method are recited unless the context indicates otherwise.

It will be understood that any component defined herein as being included can be explicitly excluded by way of proviso or negative limitation, such as any specific compounds or method steps, whether implicitly or explicitly defined herein.

Although any methods and materials similar or equivalent to those described herein can also be used in the practice or testing of the present disclosure, examples of methods and materials are now described.

### II. Proteins

The present inventors have identified mutations to platelet factor 4 (PF4) protein that reduce the strength of its binding to vaccine-induced immune thrombotic thrombocytopenia (VITT) antibodies as compared to heparin-induced thrombocytopenia (HIT) antibodies, and mutations in PF4 protein that reduce the strength of its binding to non-HIT antibodies as compared to HIT antibodies, thereby providing a way to differentiate between VITT, HIT, and non-HIT in order to optimize patient treatment.

Accordingly, in one aspect, the present disclosure provides a mutant platelet factor 4 (PF4) protein comprising a mutation to at least one amino acid selected from position R20, H23, T25, E28, K46, R49, K50, K62, K65, or K66 of the amino acid sequence of SEQ ID NO:1 (Table 1).

PF4 or platelet factor 4, also called chemokine (C-X-C motif) ligand 4 (CXCL4), as used herein refers to a small chemokine of the CXC family of chemokines with a high affinity to heparin. The wild-type PF4 protein comprises the amino acid sequence of SEQ ID NO:1.

The term “mutant PF4 protein” as used herein refers to a PF4 protein that has a mutation, such as a substitution, deletion, insertion, and translocation, compared to the wild-type PF4 protein.

The mutant PF4 proteins can be generated by any method known in the art, for example using site-directed mutagenesis or chemical synthesis or any other method of genetic engineering.

In an embodiment, the mutant PF4 protein comprises substitution of one or more mutations selected from R20A, H23A, T25A, E28A, K46A, R49A, K50A, K62A, K65A, or K66A to the amino acid sequence of SEQ ID NO:1. The mutant PF4 protein can comprise other amino acid substitutions at positions R20, H23, T25, E28, K46, R49, K50, K62, K65, or K66 of the amino acid sequence of SEQ ID NO:1, such as a substitution to valine instead of alanine. Accordingly, in another embodiment, the mutant PF4 protein comprises substitution R20V, H23V, T25V, T26V, E28V, K46V, R49V, K5OV, K62V, K65V, or K66V to the amino acid sequence of SEQ ID NO:1.

In an embodiment, the mutant PF4 protein:
(a) selectively binds a heparin-induced thrombocytopenia (HIT) antibody compared to a vaccine-induced immune thrombotic thrombocytopenia (VITT) antibody (PF4-H⁺V⁻),
(b) selectively binds the HIT antibody compared to a non-HIT antibody (PF4-H⁺NH⁻), and/or
(c) selectively binds the HIT antibody compared to both the VITT antibody and the non-HIT antibody (PF4-H⁺V⁻NH⁻).

The term “selectively binds” as used herein means that there is larger relative binding of a mutant PF4 protein herein disclosed to a first antibody than its relative binding to a second antibody. The “relative binding” or “ratio” is obtained by normalizing a quantitative measure of the binding between the mutant PF4 protein and an antibody, to the quantitative measure of the binding between a wild-type PF4 protein comprising SEQ ID NO:1 to the same antibody. For example, when the relative binding of a mutant PF4 protein to the HIT antibody when normalized to the binding of the wild-type PF4 protein to the HIT antibody is larger than the relative binding of the same mutant PF4 protein to the VITT antibody when normalized to the binding of the wild-type PF4 protein to the VITT antibody, the mutant PF4 protein is selective for HIT. In an embodiment, the relative binding is 5%, 10%, 15%, 20%, 25%, 30%, 35%, 40%, 45%, 50%, 55%, 60%, 65%, 70%, 75%, 80%, 85%, 90%, 95%, 100%, 105%, 110%, or more.

Binding can also be described in terms of “binding affinity”. The term “affinity”, as used herein, refers to the strength of the sum total of noncovalent interactions between a single binding site of a molecule and its binding partner. Unless indicated otherwise, as used herein, “binding affinity” refers to intrinsic binding affinity which reflects a 1:1 interaction between members of a binding pair. The affinity of a molecule X for its partner Y can generally be represented by the equilibrium dissociation constant (K_{D}). Affinity can be measured by common methods known in the art.

The term “heparin-induced thrombocytopenia” or “HIT” as used herein means an antibody-mediated drug disorder arising in patients receiving heparin as an anticoagulant medication, often following major surgical procedures. Accordingly, the term “heparin-induced thrombocytopenia antibody” or “HIT antibody” as used herein means an anti-PF4 or anti-PF4/heparin antibody that is produced in a subject with HIT.

The term “vaccine-induced immune thrombotic thrombocytopenia” or “VITT” as used herein means a rare but serious adverse event associated with adenoviral-vector based vaccines for SARS-CoV-2 which resembles, but is separate from, HIT. Accordingly, the term “vaccine-induced immune thrombotic thrombocytopenia antibody” or “VITT antibody” as used herein means an anti-PF4 antibody that is produced in a subject with VITT.

The term “non-heparin-induced thrombocytopenia” or “non-HIT” as used herein means refers to those patients suspected of having HIT based on their clinical symptoms and develop anti-PF4/heparin antibodies, yet test negative for platelet-activating antibodies in functional assays. Accordingly, the term “non-heparin-induced thrombocytopenia antibody” or “non-HIT antibody” as used herein refers to an anti-PF4 or anti-PF4/heparin antibody that is produced in a subject with non-HIT.

The term “antibody” as used herein is intended to include monoclonal antibodies, polyclonal antibodies, chimeric antibodies, and humanized antibodies. The antibody can be from recombinant sources and/or produced in transgenic animals. Also included are dimers, minibodies, diabodies, and multimers thereof, bi-specific and multi-specific antibody fragments, and domain antibodies.

The term “PF4-H⁺V⁻” as used herein mean a mutant PF4 protein that selectively binds a HIT antibody compared to a VITT antibody.

The term “PF4-H⁺NH⁻” as used herein means a mutant PF4 protein that selectively binds a HIT antibody compared to a non-HIT antibody.

The term “PF4-H⁺V⁻NH⁻” as used herein means a mutant PF4 protein that selectively binds a HIT antibody over both a VITT antibody and a non-HIT antibody. The terms PF4-H⁺V⁻, PF4-H⁺NH⁻, and PF4-H⁺V⁻ are not mutually exclusive. A mutant PF4 protein classified as PF4-H⁺V⁻NH⁻ is also by definition a PF4-H⁺V⁻ and PF4-H⁺NH⁻.

In an embodiment, the PF4-H⁺V⁻ comprises the substitution R20A, H23A, T25A, E28A, K46A, R49A, K50A, K62A, K65A, or K66A to the amino acid sequence of SEQ ID NO:1. In another embodiment, the PF4-H⁺V⁻ comprises the substitution H23A, E28A, K50A, K62A, or K66A to the amino acid sequence of SEQ ID NO:1. In yet another embodiment, the PF4-H⁺V⁻ comprises the substitution H23A, E28A, K50A, or K66A to the amino acid sequence of SEQ ID NO:1.

In an embodiment, the PF4-H⁺NH⁻ comprises the substitution H23A, T25A, R49A, K65A, or K66A to the amino acid sequence of SEQ ID NO:1. In another embodiment, the PF4-H⁺NH⁻ comprises the substitution H23A, T25A, or K65A to the amino acid sequence of SEQ ID NO:1.

In an embodiment, the PF4-H⁺V⁻NH⁻ comprises the substitution H23A, T25A R49A, K65A, or K66A to the amino acid sequence of SEQ ID NO:1.

### III. Methods

The present inventors have also identified methods of differentiating between heparin-induced thrombocytopenia (HIT), vaccine-induced immune thrombotic thrombocytopenia (VITT), and non-HIT using the mutant platelet factor 4 (PF4) proteins described herein.

Accordingly, in one aspect, the present disclosure provides a method of differentiating between VITT, HIT, and non-HIT in a sample from a patient suspected of having VITT, HIT or non-HIT comprising:
a) contacting the sample with at least one of the mutant PF4 protein herein disclosed and measuring binding of the at least one mutant PF4 protein with the sample;
b) contacting the sample with the PF4 protein comprising the amino acid sequence of SEQ ID NO:1 and measuring the binding of the PF4 protein with the sample;
c) determining a ratio of the binding in a) to the binding in b); and
d) determining the level of similarity of the ratio in c) to one or more control profiles, wherein (i) a high level of similarity to a HIT specific control profile or a low level of similarity to a VITT specific control profile and/or a non-HIT specific control profile is indicative of HIT; (ii) a high level of similarity to the VITT specific control profile or a low level of similarity to the HIT specific control profile and/or the non-HIT specific control profile is indicative of VITT; and/or (iii) a high level of similarity to the non-HIT specific control profile or a low level of similarity to the VITT specific control profile and/or the HIT specific control profile is indicative of non-HIT.

The term “contacting” as used herein refers to bringing the disclosed PF4 protein or mutant and the sample together in such a manner that the PF4 protein or mutant can interact with a target protein in the sample, for example a HIT antibody, a VIT antibody, and/or a non-HIT antibody.

The PF4 protein or mutant herein disclosed can be immobilized on a surface in order to measure its binding to the sample. Accordingly, in an embodiment, the method first comprises the step of immobilizing the mutant PF4 protein in a) and/or the PF4 protein in b) on a support, optionally a biosensor tip or microplate.

The term “immobilizing” or “immobilized’ as used herein means at least attaching a PF4 protein or mutant herein disclosed to a surface, for example a support. The PF4 protein or mutant can be immobilized to the surface using adsorption techniques including non-covalent interactions (e.g., electrostatic forces, van der Waals, and dehydration of hydrophobic interfaces) and covalent binding techniques where functional groups or linkers facilitate attaching the biomolecules to the surface. Immobilizing the PF4 protein or mutant to the surface may be based upon the properties of the surface or the properties of the PF4 proteins or mutants themselves. In some cases, a surface may be functionalized (e.g., chemically or physically modified) to facilitate immobilizing the PF4 proteins or mutants to the surface.

The term “support” as used herein means any material which is capable of binding a PF4 protein or mutant herein disclosed. Supports can be biosensor tips, ELISA supports, matrices, columns, coverslips, chromatographic materials, filters, microscope slides, test tubes, vials, bottles, glass or plastic surfaces, such as a microplate, sheets, particles, and beads, including magnetic beads.

The PF4 protein or mutant herein disclosed can be immobilized to the surface, optionally a support or a biosensor tip, using avidin/streptavidin-biotin interaction. Accordingly, in an embodiment, the mutant PF4 protein in a) and/or the PF4 protein in b) is biotinylated.

Heparin, or unfractionated heparin, is a naturally occurring large anionic polysaccharide that can cluster PF4 tetramers thereby revealing neoepitopes. The neoepitopes can be recognized by IgG-specific antibodies that bind to PF4/heparin and form immunocomplexes. Mutations to the PF4 protein may impact its binding affinity to heparin and/or its binding affinity to an antibody, such as the HIT antibody, the VITT antibody, or the non-HIT antibody, that recognizes the PF4 protein, and/or PF4/heparin. Accordingly, in an embodiment, the mutant PF4 protein in a) and/or the PF4 protein in b) is immobilized alone or complexed with unfractionated heparin.

The measuring binding in step a) and/or b) can comprise any method known in the art to measure protein binding. Accordingly, in an embodiment, the method comprises enzyme-linked immunosorbent assay (ELISA), enzyme immunoassay (EIA), or bio-layer interferometry (BLI).

ELISA and EIA can comprise:
1. immobilizing the PF4 protein or mutant to a surface such as a microplate surface;
2. adding a patient sample to the surface and incubating the patient sample with the immobilized PF4 protein or mutant;
3. washing the surface to remove unbound antibodies;
4. adding a secondary antibody with a detectable tag, optionally a fluorescent tag, directed against antibodies from the patient, such as an anti-human IgG; and
5. detecting the amount of binding between the antibodies present in the patient sample and the PF4 protein or mutant through detection of the bound secondary antibody.

BLI can comprise:
1. Immobilizing the PF4 protein or mutant on a biosensor tip;
2. Contacting the biosensor tip with a patient sample; and
3. Measuring a shift in optical interference pattern (observed as a colorimetric shift) induced by the binding of the HIT antibody, the VITT antibody, and/or the non-HIT antibody to the PF4 protein or mutant.

Accordingly, in an embodiment, the measuring in step a) and/or step b) comprises measuring the detection of a secondary antibody tagged with a detectable label against the HIT antibody, the VITT antibody, and/or the non-HIT antibody, for example, by detection of the presence of human antibodies using anti-human IgG. In an embodiment, the detectable label is a fluorescent label. In another embodiment, the detectable label is a colorimetric label. In another embodiment, the measuring in step a) and/or step b) comprises measuring a colorimetric shift in optical interference pattern induced by binding of the HIT antibody, the VITT antibody, and/or the non-HIT antibody to the mutant PF4 protein or the PF4 protein comprising the amino acid sequence of SEQ ID NO:1.

In another aspect, the present disclosure provides a method of differentiating between VITT and HIT in a sample from a patient suspected of having VITT or HIT comprising:
I) combining the sample with platelets labeled with serotonin;
II) incubating the combined sample and platelets with no unfractionated heparin and at least two concentrations of the unfractionated heparin and measuring serotonin release to the no unfractionated heparin and to the at least two concentrations of the unfractionated heparin;
III) incubating the combined sample and platelets with no mutant PF4 protein and at least two concentrations of the mutant PF4 protein herein disclosed, and measuring the serotonin release to the no mutant PF4 protein and to the at least two concentrations of the mutant PF4 protein;
IV) measuring a dose response of the sample in II) and in III); and
V) determining the level of similarity of the dose response in IV) to one or more control profiles, wherein (i) a high level of similarity to a HIT specific control profile or a low level of similarity to a VITT specific control profile is indicative of HIT; and/or (ii) a high level of similarity to the VITT specific control profile or a low level of similarity to the HIT specific control profile is indicative of VITT.

Platelet-rich plasma can be prepared, and the platelets labeled by any method known in the art, for example using ¹⁴C-serotonin as described in Nazy et al., 2015. In an embodiment, the platelets are labelled with ¹⁴C-serotonin. Serotonin release can be measured by any method known in the art, and optionally a scintillation counter.

Suitable concentrations of the unfractionated heparin can comprise pharmacologic and high concentrations, for example 0.1 U/mL, 0.2 U/mL, 0.3 U/ml, 0.4 U/mL, 0.5 U/mL, 1 U/mL, 5 U/mL, 10 U/mL, 25 U/mL, 50 U/mL, 75 U/mL, 100 U/mL, or more.

Suitable concentrations of the mutant PF4 protein can comprise 5μg/mL, 10 μg/mL, 15 μg/mL, 20 μg/mL, 25 μg/mL, 30 μg/mL, 35 μg/mL, 40 μg/mL, 45 μg/mL, 50 μg/mL, 60 μg/mL, 70 μg/mL, 80 μg/mL, 90 μg/mL, 100 μg/mL or more.

The term “dose response” as used herein means a relationship between the dose of unfractionated heparin and/or PF4 protein or mutants provided to the sample, and the magnitude of the serotonin release response. Accordingly, the term “non-null dose response” as used herein means that there is a relationship between the dosage of unfractionated heparin and/or mutant PF4 protein and the magnitude of the serotonin release response, indicating that the response is at least partially dependent on the presence and/or dosage of the unfractionated heparin and/or mutant PF4 protein. Likewise, the term “null dose response” as used herein means that there is no relationship between the dosage of the unfractionated heparin and/or mutant PF4 protein and the magnitude of the serotonin release response, indicating that the response is independent of the unfractionated heparin and/or mutant PF4 protein.

The antibodies that bind to the mutant PF4 protein and/or to PF4/heparin to form immunocomplexes activate platelets through their FcγRIIa receptors, resulting in serotonin release. If these immunocomplexes are involved in the observed serotonin release, serotonin release can be prevented by blocking these FcγRIIa receptors. Accordingly, in an embodiment, the platelets in I) are preincubated with FcγRIIa-blocking monoclonal antibody (IV.3). IV.3 can be from any organism, and suitable examples are commercially available, for example, Anti-Human CD32 Antibody Clone IV.3, catalog # 60012 from STEMCELL Technologies, PE Anti-human CD32 Antibody *IV.3*, product # 103201L0 from AAT Bioquest, or InVivoMAb anti-human CD32 (FcγRIIA), catalog #BE0224 from BioXCell.

In an embodiment, the HIT specific control profile comprises a non-null dose response to both i) the unfractionated heparin, and ii) the mutant PF4 protein; and/or wherein the VITT specific control profile comprises a null dose response to i) the unfractionated heparin, and the non-null dose response to ii) the mutant PF4 protein.

In an embodiment, the sample is a blood sample, optionally a serum or plasma sample.

In an embodiment, a high level of similarity to the HIT specific control profile is indicated by a higher correlation value computed between the sample profile and the HIT specific control profile than an equivalent correlation value computed between the sample profile and the VITT specific control profile and/or the non-HIT specific control profile, optionally wherein the correlation value is a correlation coefficient.

In another embodiment, a high level of similarity to the VITT specific control profile is indicated by a higher correlation value computed between the sample profile and the VITT specific control profile than an equivalent correlation value computed between the sample profile and the HIT specific control profile and/or the non-HIT specific control profile, optionally wherein the correlation value is a correlation coefficient.

In yet another embodiment, a high level of similarity to the non-HIT specific control profile is indicated by a higher correlation value computed between the sample profile and the non-HIT specific control profile than an equivalent correlation value computed between the sample profile and the HIT specific control profile and/or the VITT specific control profile, optionally wherein the correlation value is a correlation coefficient.

Methods of determining the similarity between profiles are well known in the art. Methods of determining similarity may in some embodiments provide a non-quantitative measure of similarity, for example, using visual clustering. In other embodiments, similarity may be determined using methods which provide a quantitative measure of similarity.

In an embodiment, similarity may be measured by computing a “correlation coefficient”, which is a measure of the interdependence of random variables that ranges in value from −1 to +1, indicating perfect negative correlation at −1, absence of correlation at zero, and perfect positive correlation at +1. In an embodiment, the correlation coefficient may be a linear correlation coefficient, for example, a Pearson product-moment correlation coefficient.

A Pearson correlation coefficient (r) is calculated using the following formula:

$$r = \frac{{\sum }_{i} ( {x}_{i} - \overline{x} ) ( {y}_{i} - \overline{y} )}{\sqrt{{\sum }_{i} {( {x}_{i} - \overline{x} )}^{2}} \sqrt{{\sum }_{i} {( {y}_{i} - \overline{y} )}^{2}}}$$

In an embodiment, the correlation coefficient may be a monotonic correlation coefficient, for example, a Spearman's rank correlation coefficient.

A Spearman's rank correlation coefficient (ρ) is calculated using the following formula:

$${r}_{s} = {ρ}_{R ( X ) , R ( Y )} = \frac{cov ( R ( X ) , R ( Y ) )}{{σ}_{R ( X )} {σ}_{R ( Y )}}$$

where cov(R(X),R(Y)) is the covariance of rank variables and σ_{R(X)} and σ_{R(Y)} are their standard deviations.

In one embodiment, x and y are the measures of binding strength in a sample profile and a control profile, respectively.

The control profile may be a reference value and/or may be derived from one or more samples, optionally from historical HIT, VITT, and/or non-HIT data from a pool of samples with known annotation of HIT, VITT, and/or non-HIT. In an embodiment, the control profile is a value that is continually updated as further samples are collected and selective and/or relative antibody binding and/or serotonin release dose response to unfractionated heparin and/or mutant PF4 protein are measured and correlated. It will be understood that the control profile represents an average of the values for the selective and/or relative antibody binding and/or serotonin release dose response to unfractionated heparin and/or mutant PF4 protein described herein. Average values may, for example, be the mean values or median values. In an embodiment, the control profile may be obtained experimentally.

In an embodiment, a correlation coefficient calculated between a sample profile and a control profile indicates a high level of similarity to the control profile when the correlation coefficient has an absolute value between 0.5 to 1, optionally between 0.75 to 1, and a low level of similarity to the control profile when the correlation coefficient has an absolute value between 0 to 0.5, optionally between 0 to 0.25.

It will be appreciated that any “correlation value” which provides a quantitative scaling measure of similarity between profiles may be used to measure similarity.

The present disclosure also provides the following embodiments:

Embodiment 1. A mutant Platelet Factor 4 (PF4) protein comprising one or more mutations in the heparin binding region of the PF4 tetramer with respect to a wild-type PF4 protein.

Embodiment 2. The mutant PF4 protein of embodiment 1 comprising one or more mutations at positions selected from R22, R20, H23, E28, K46, N47, K50, K62, K66, T26, T25, R49, R46, K65 with respect to a wild-type PF4 protein.

Embodiment 3. The mutant PF4 protein of embodiment 1 or 2, comprising one or more mutations selected from R22A, H23A, E28A, K46A, N47A, K50A, K62A, R20A, T25A, R46A, R49A, K65A, or K66A.

Embodiment 4. The mutant protein of any one of embodiments 1-3 wherein the mutant protein binds to vaccine-induced immune thrombotic thrombocytopenia (VITT) antibodies with a different affinity relative to heparin-induced thrombocytopenia (HIT) antibodies or non-HIT antibodies, wherein the mutant PF4 proteins that bind to VITT antibodies with a different affinity relative to HIT antibodies comprise H23A, E28A, K62A, K50A, K66A, R20A, T26A, K46A, R49A, K65A and the mutant PF4 proteins that bind to HIT antibodies with a different affinity relative to non-HIT antibodies comprise R20A, T25A, R46A, R49A, K65A, K66A.

Embodiment 5. A method for the differentiation of VITT from HIT and/or HIT from non-HIT in a patient sample comprising a Serotonin Release Assay (SRA) or a binding assay including but not limited to Enzyme-linked Immunosorbent Assay (ELISA), Bio-Layer Interferometry (BLI).

Embodiment 6. An ELISA method of Embodiment 5 for the differentiation of VITT and HIT through the detection of VITT or HIT antibodies in a patient sample, the method comprising:
a) Immobilizing the one or more mutant proteins of any of embodiments 1-3 or wild-type PF4;
b) Blocking the unbound sites;
c) Adding a patient sample and incubating for a period of time to allow antibodies present in the sample to bind to the mutant PF4 protein or proteins;
d) Washing away unbound antibodies;
e) Adding a secondary antibody directed against the patient sample antibodies; and
f) Quantifying the binding level of antibodies present in the patient sample to the mutant PF4 through colorimetric or fluorescent detection systems;
   wherein a difference or similarity between the binding level in a patient sample compared to a HIT or healthy sample control is indicative of the presence or absence of VITT or HIT

Embodiment 7. The method of embodiment 6, wherein VITT, HIT, or non-HIT can be diagnosed when the antibody binding in a sample to one or more PF4 mutants is reduced compared to i) the antibody binding of the sample to wild-type PF4, and ii) the binding of a HIT control sample to the one or more mutants and wild-type PF4.

Embodiment 8. A BLI method of embodiment 5 for the differentiation of VITT and HIT, and HIT from non-HIT, the method comprising:
a) biotinylation of the mutants of claims 1-3 and wild type PF4;
b) immobilizing biotinylated recombinant PF4 alone or complexed with unfractionated heparin on biosensor tips;
c) reacting the antigen-coated biosensor tips with a patient sample; and
d) measuring binding profile response of each patient sample;
   wherein patient samples tested by BLI results in higher binding and avidity responses for VITT or HIT samples compared to i) the binding and avidity responses in HIT control samples with both PF4 and PF4/heparin and ii) the binding and avidity responses in healthy controls.

Embodiment 9. The serotonin release assay (SRA) method of embodiment 5 for the differentiation of HIT and VITT or HIT and non-HIT through the measurement of platelet activation, the method comprising the standard (SRA) with heparin and a modified SRA to increase doses of PF4 (PF4-SRA) rather than heparin wherein patient samples tested by SRA show atypical reactivity in the absence or presence of heparin and strong dose response with the addition of PF4 in VITT patients, whereas HIT patients show heparin dependent platelet activation in the presence of heparin or PF4.

Embodiment 10. The method of embodiments 5-9 wherein the patient sample is a blood sample, optionally, a serum or plasma sample.

Embodiment 11. A method of reducing the severity and progression of VITT, comprising blocking PF4 in the heparin binding region.

Embodiment 12. The method of embodiment 11, wherein the blocking agent for PF4 comprises an aptamer, small molecule or an antibody fragment of a monoclonal antibody for the heparin binding region of PF4.

The above disclosure generally describes the present invention. A more complete understanding can be obtained by reference to the following specific examples. These examples are described solely for the purpose of illustration and are not intended to limit the scope of the invention. Changes in form and substitution of equivalents are contemplated as circumstances might suggest or render expedient. Although specific terms have been employed herein, such terms are intended in a descriptive sense and not for purposes of limitation.

### EXAMPLES

The following non-limiting examples are illustrative of the present disclosure:

### Example 1. Results and Discussion

Patient Demographics

Sera from patients with VITT (n=5) were referred to the McMaster Platelet Immunology Laboratory for confirmatory diagnosis. All VITT patients had received a single dose of the ChAdOx1 nCoV-19 vaccine (AstraZeneca COVID-19 Vaccine, AstraZeneca; COVIDSHIELD, Verity Pharmaceuticals and Serum Institute of India) and subsequently developed thrombocytopenia and thrombosis. All VITT sera had antibodies against PF4. Sera from patients with HIT (n=10) were used as comparators. HIT patients had thrombocytopenia and thrombosis after receiving heparin and had a high clinical probability score (4T score ³4)15 with detectable anti-PF4/heparin antibodies and heparin-dependent platelet activation. VITT patients (n=5) had a mean age of 44 years (range: 35-72) and 2/5 were female. The time from first dose of the ChAdOx1 nCoV-19 vaccine to sample collection 14 to 40 days (mean 28 days). All VITT sera had antibodies against PF4 (mean OD: 2.71, range: 0.763-3.347). HIT patients (n=10) had a median age of 69 years (range: 52-81 years) and 5/10 were female. Nine of 10 HIT patients (90%) experienced thrombosis. The time from heparin initiation to sera collection was 6-27 days (mean 14.3 days). All HIT patients had detectable anti-PF4/heparin antibodies (mean OD: 3.10, range: 2.329-3.897).

Platelet Activation Profiles of VITT Antibodies

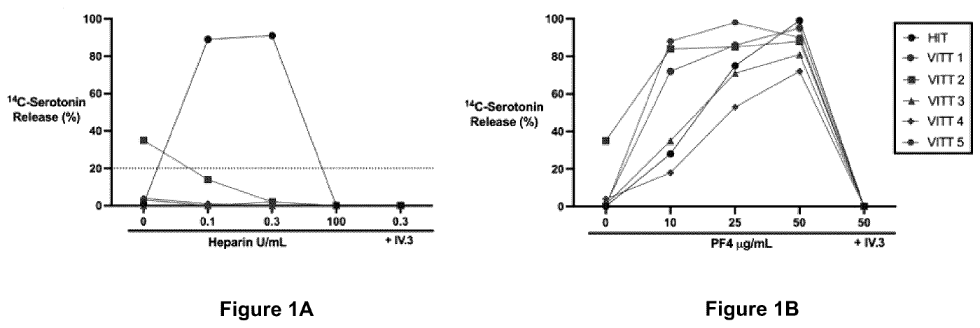

Using a functional platelet activation assay [serotonin release assay (SRA)], it was shown that sera from VITT and HIT patients had distinctly different patterns of reactivity. There was no consistent pattern of heparin-dependent reactivity with the VITT sera (FIG. 1A). In contrast, all HIT sera had a characteristic heparin-dependent pattern of platelet reactivity. All 5 VITT sera demonstrated strong and dose-dependent PF4-mediated platelet activation (81% to 98% release with 50 μg/mL of PF4) (FIG. 1B). Complete inhibition of platelet activation by HIT and VITT was achieved with the addition of the FcγRIIa blocking monoclonal antibody IV.3. These results indicate that VITT antibodies require PF4 for platelet activation, which is mediated by engagement of FcγRIIa receptors consistent with an immune complex. Furthermore, the lack of heparin dependency of VITT antibodies for the activation of platelets, yet inhibition by 0.3 U/mL of heparin of VITT-mediated platelet activation, in VITT sample #2 (FIG. 1B), indirectly suggested that VITT could bind to the heparin binding region on PF4, which was directly investigated, below, using epitope-mapping.

Binding Region of VITT Antibodies on PF4

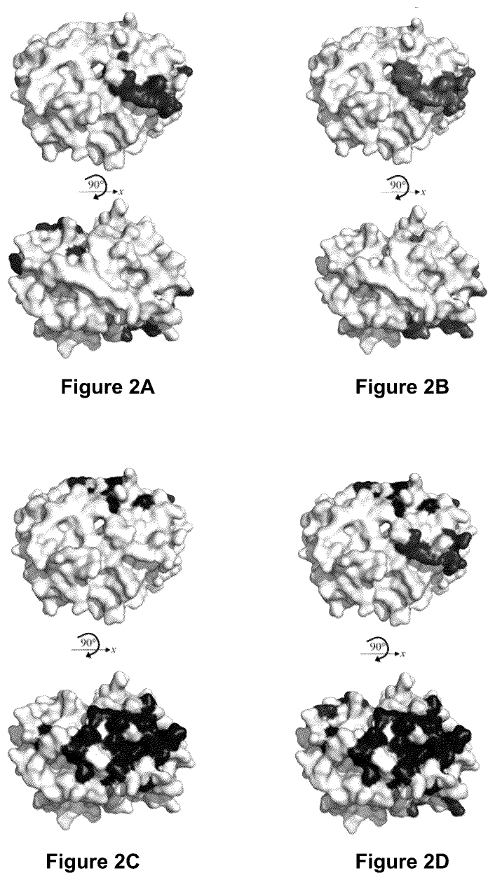

Previous studies using linear peptides of PF4 have shown that the binding epitopes of HIT antibodies are non-contiguous conformational epitopes, and the absence of tetrameric PF4 in these peptides is not useful for epitope mapping studies¹⁶. Instead, alanine scanning mutagenesis was previously used to identify those PF4 amino acids required for binding HIT antibodies and showed that the tetrameric nature of PF4 is preserved and more suitable for epitope mapping in both binding⁷ and functional studies¹⁷. Therefore, to identify the specific amino acid targets of VITT antibodies on PF4, alanine scanning mutagenesis was used to produce 70 unique recombinant PF4 mutants, each differing by a single amino acid⁷. A critical binding amino acid on PF4 was defined as one that caused a greater than 50% reduction in binding compared to wildtype PF4. 8 surface amino acids were identified that were necessary for binding of all VITT sera (R22A, H23A, E28A, K46A, N47A, K50A, K62A, and K66A). This restricted epitope is consistent with limited B-cell clonality and suggests that VITT antibodies bind to a specific region on PF4. Using PyMOL molecular visualization platform, it was observed that the 8 amino acids corresponded to the heparin binding region on the PF4 tetramer (FIG. 2A and FIG. 2B). In fact, 4/8 amino acids that are part of the VITT epitope (R22A, H23A, K46A, and K66A) are also binding amino acids of heparin. It can be postulated that the binding of the VITT anti-PF4 antibodies to the heparin binding region on the PF4 tetramer explains why heparin concentrations inhibit platelet activation, presumably by displacing the VITT anti-PF4 antibodies. In addition, this binding region permits the VITT anti-PF4 antibodies to function similarly to heparin by clustering the PF4, thus permitting crosslinking of PF4 tetramers into immune complexes. These results explain why some VITT sera tested in studies by Schultz et al² and Greinacher et al¹ were inhibited by therapeutic doses of heparin. In addition, VITT antibodies bind to a similar region on PF4 as the monoclonal antibody 1E12, which is also known to activate platelets independent of heparin¹⁸ by crosslinking PF4 tetramers to create platelet-activating immune complexes. These studies suggest that VITT antibodies cause platelet activation through a similar heparin-like mechanism by stabilizing complexes of PF4, aligning the Fc-portion in close proximity and crosslinking FcγRIIa receptors on platelets.

The VITT binding region on PF4 was compared with 10 HIT sera. No single common amino acid was critical for binding of all 10 HIT sera, likely due to the polyclonal nature of the antibodies, as previously shown⁷. When all the critical amino acids from screening the 10 HIT patients were combined, there was a total of 10 amino acids (L8A, C10A, C12A, T16A, R22A, Q40A, N47A, C52A, L53A, D54A, K61A, K66A, L67A) that were part of the various HIT epitopes, in different combinations. PF4 mutants L8A, C10A, C12A, T16A and K61A were the most common amino acids to affect the binding of HIT sera and they were only common among 6 of the HIT sera. When these amino acids were displayed on the PF4 tetramer, one region on PF4 was identified that all (10/10) HIT antibodies targeted (FIG. 2C and FIG. 2D, blue colour). This region was distant from the heparin binding region. In addition, 6/10 of the HIT antibodies targeted an additional region (FIG. 2D), which was similar to the VITT region and within the heparin binding region. Unlike VITT sera, none of the HIT sera were restricted to the heparin binding region only. This is consistent with previous observation that some HIT sera contain two types of platelet-activating characteristics both heparin-dependent and heparin-independent, simultaneously¹⁹. In contrast, VITT antibodies only had heparin-independent antibodies. HIT antibodies bound to a similar region as the KKO, a monoclonal antibody against PF4/heparin complexes, thus providing an explanation as to why HIT antibodies, but not VITT antibodies, require heparin to crosslink PF4 tetramers⁷.

In addition to clarifying the amino acid targets of VITT antibodies, these results provide an explanation for why some rapid HIT immunoassays^{20,21} may yield false-negative results for VITT. One of these assays, the latex immunoturbidimetric assay [HemosIL® HIT-Ab(PF4-H)] uses KKO to aggregate complexes of PF4/heparin. Since all HIT sera have antibodies that bind to the same region as KKO, the addition of HIT to this test competes with KKO in binding the PF4/heparin complexes. In contrast, VITT antibodies bind to a different region on PF4 than KKO, and thus do not compete for binding.

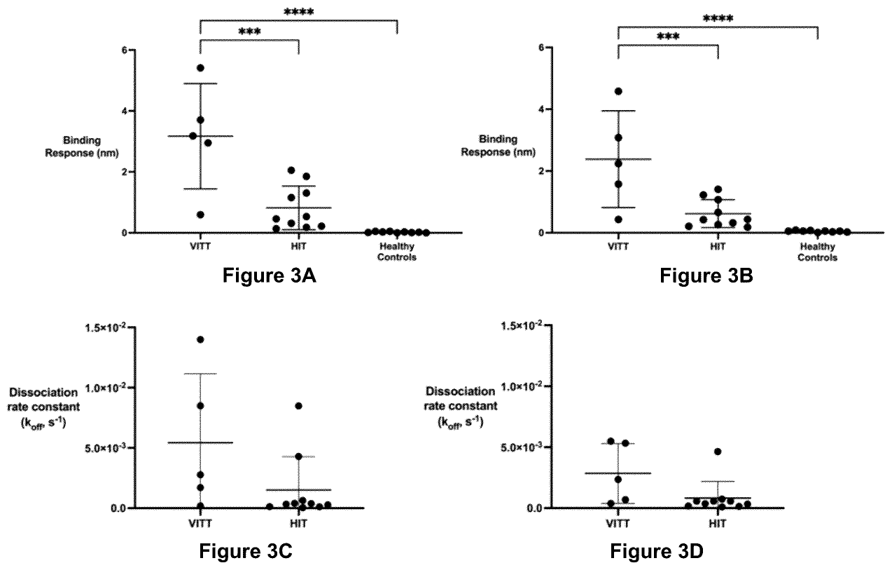

Binding kinetics of VITT antibodies: Bio-layer interferometry (BLI) was used to measure the binding characteristics of antibodies from patient sera. The binding response of VITT sera (n=5), HIT sera (n=10) and sera from healthy controls (n=10) to PF4 and PF4/heparin were tested using BLI. When sera were tested with immobilized PF4, the mean binding response [nm shift±standard deviation (SD)] was 3.17±1.73 nm for VITT patients; 0.82±0.72 nm for HIT patients; and 0.0059±0.055 nm for healthy controls (FIG. 3A). Similarly, when sera were tested with immobilized PF4/heparin complexes, the mean binding response±SD was 2.38±1.57 nm for VITT patients, 0.62±0.45 nm for HIT patients and 0.035±0.053 nm for healthy controls (FIG. 3B). The binding response in VITT sera was significantly higher than HIT sera and healthy controls with both PF4 and PF4/heparin (VITT vs. HIT: p<0.005, VITT vs. healthy controls: p<0.001). Binding responses are a measure of abundance of antigen-specific antibodies present in a given sample²². Therefore, VITT patients have the highest binding response in BLI when compared to antibodies found in HIT patients.

In the case of polyclonal antibodies interacting with an antigen, the dissociation rate, which is concentration independent, can be measured. The measured dissociation rate is an average of dissociation rates of many antibodies that are simultaneously dissociating from an antigen²². The dissociation rate reflects the avidity of an antibody for a given antigen²³. The dissociation rates of VITT and HIT sera was measured and found the mean dissociation rate (k_{off} s⁻¹±SD) was 5.44×10-3±0.0057 s⁻¹ for VITT patients and 1.51×10-3±0.0028 s−1 for HIT patients with immobilized PF4 (FIG. 3C). Similarly, the mean dissociation rate (k_{off} s⁻¹±SD) was 2.85×10-3±0.0024 s⁻¹ for VITT patients and 8.24×10-4±0.0014 s⁻¹ for HIT patients with immobilized PF4/heparin (FIG. 3D). There was no statistically significant difference in dissociation rates between the two groups with both PF4 and PF4/heparin (p=0.091 and p=0.057, respectively). This indicates the avidity of antibodies is similar in both groups. As antibodies from human sera are polyclonal, BLI was only able to calculate binding responses and dissociation rates, which can provide information on the strength of the immune response and the avidity of the polyclonal sera, respectively; however, the affinity could not be determined.

Previous studies have shown that monoclonal antibodies against PF4, such as KKO²⁴ and 1E12¹⁸ facilitate the formation of ultra-large and stable complexes of PF4 on the platelet surface. With a higher binding response than HIT antibodies, albeit with similar avidity, VITT antibodies are likely capable of creating the same ultra-large complexes of PF4 leading to the subsequent platelet activation in the absence of heparin. Given the increased level of binding responses in VITT patients compared to HIT patients but similar dissociation rates, this suggests that the antibody response is stronger in VITT patients.

Developing an ELISA that can Separate VITT from HIT

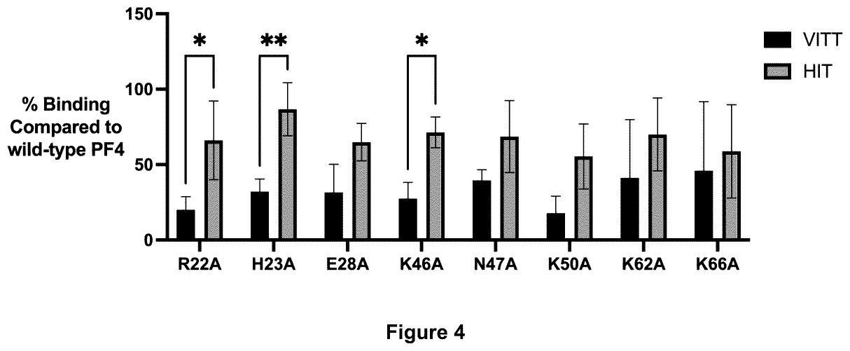

Based on the epitope mapping data obtained in this report, the 8 PF4 mutants (R22A, H23A, E28A, K46A, N47A, K50A, K62A, and K66A) that affected the binding of VITT samples (n=3) were tested and compared their reactivities to HIT patient sera (n=10). Three PF4 mutants (R22A, H23A, and K46A) in the ELISA format were able to show a significant difference in binding between VITT patient sera from HIT (R22A p<0.05, H23A p<0.01, K46A p<0.05). In contrast, the other five PF4 mutants (E28A, N47A, K50A, K62A, and K66A) did not show a significant separation between the VITT and HIT patient sera tested (FIG. 4).

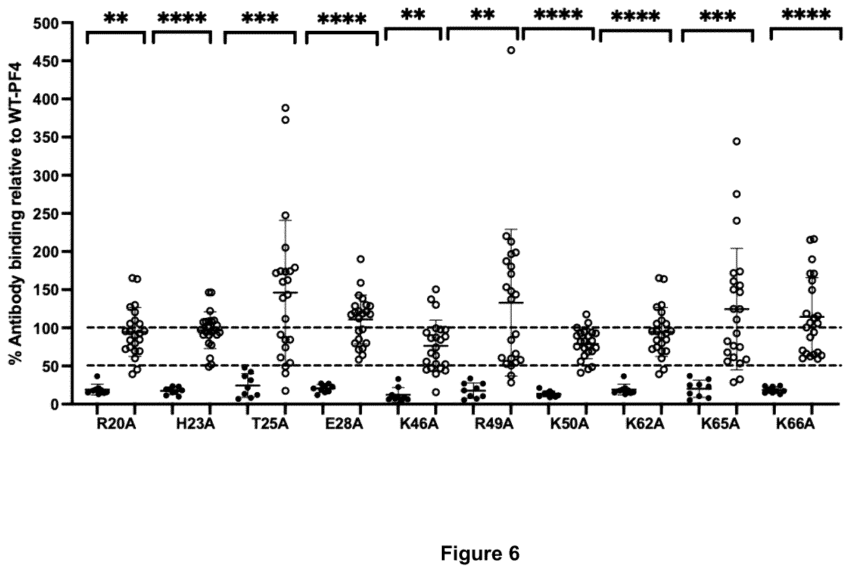

Screening for PF4 mutants that would be used to separate HIT from VITT was based on previous data that revealed the amino acids that make up heparin binding site (R20, R22, H23, T25, K46, R49, K61, K62, K65, K66) 31 and the VITT site (R22, H23, E28, K46, N47, K60, K62, K66)³² on PF4. The amino acids that make up the HIT binding sites as previously published and confirmed in this study by mapping the binding sites of HIT patient samples and HIT-like monoclonal antibodies were excluded during the selection process. This was done to avoid selecting PF4 mutants that consistently resulted in a loss of binding against the HIT patient samples and against the HIT-like monoclonal antibodies. Based on the data generated from mapping the epitopes of HIT and VITT antibodies, a total of 10 PF4 mutants were selected to be used in differentiating between HIT and VITT in an EIA (FIG. 6). All 10 selected mutants (R20A: 19.1% vs. 94.6%, p<0.05, H23A: 17.4% vs. 97.0%, p<0.0001, T25A: 24.5% vs. 146.1%, p<0.001, E28A: 20.5% vs. 111.0%, p<0.0001, K46A: 12.2% vs. 76.4%, p<0.001, R49A: 17.8% vs 132.9%, p<0.01, K50A: 13.4% vs. 79.0%, p<0.0001, K62A: 19.6% vs. 94.6%, p<0.0001, K65A: 20.1% vs. 124.5%, p<0.001, K66A: 18.5% vs. 114.4%, p<0.0001) were able to distinguish HIT from VITT antibodies with statistical significance (FIG. 6). The mutants H23A, E28A, K50A and K66A were better at separating HIT from VITT antibodies when using cut-offs of 45.5%, 42.7%, 31.1%, and 41.8%, respectively and all had high sensitivities (100%) and specificities (100%).

PF4 Mutants that Separate HIT from Non-HIT

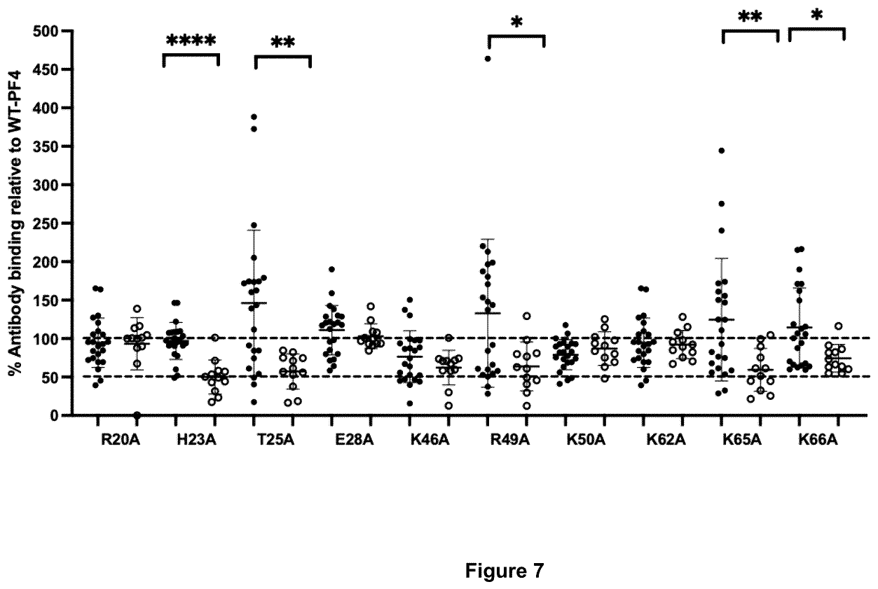

To differentiate between HIT and non-HIT, the serum samples of patients that presented with HIT-like symptoms were sent in for diagnostic testing. The serum samples from both HIT (n=22) and non-HIT (n=12) patient groups that were positive for antibodies against wild-type PF4 in our in-house IgG specific PF4/heparin EIA³³ (OD405 nm≥0.45) and the streptavidin/biotinylated-heparin/PF4 EIA (OD405 nm≥0.45) were selected for further analysis. The standard SRA⁴ was used to confirm the presence of platelet-activating antibodies; the ‘HIT’ patient cohort consisted of patients that tested positive for platelet-activating antibodies (≥20% ¹⁴C-serotonin release) and the non-HIT patient cohort consisted of patients that tested negative for platelet activating antibodies in (£20% ¹⁴C-serotonin release) following subsequent testing in the standard SRA. These HIT and non-HIT patient cohorts were then tested against the 10 previously selected PF4.mutants in an EIA. 5 out of 10 of these PF4 mutants (H23A: 96.9% vs. 50.0%, p<0.0001, T25A: 146.1% vs. 57.2%, p<0.01, R49A: 132.9% vs. 63.9%, p<0.05, K65A: 124.5% vs. 59.3%, p<0.05 and K66A: 114.4% vs. 74.4%, p<0.05)) were able to separate HIT from non-HIT with statistical significance (FIG. 7). All 5 of these amino acids are part of those that make up the heparin binding site on PF4. None of the amino acids overlap with those found in the VITT site on PF4. The mutants H23A, T25A, and K65A were better at separating HIT from non-HIT antibodies when using cut-offs of 49.8%, 58.9%, and 52.4% respectively and all had low sensitivities (50%) and high specificities of 95.7%, 82.1% and 91.3% in the EIA testing HIT and non-HIT samples against the PF4 mutants H23A, T25A and K65A, respectively.

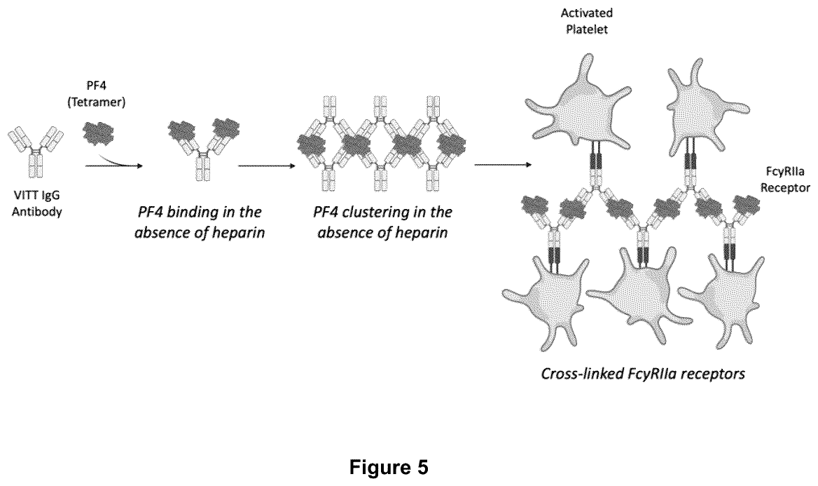

This study offers an explanation for VITT-mediated platelet activation. In rare individuals, unique antibodies form following exposure to the ChAdOx1 nCoV-19 vaccine 1-3. These antibodies are remarkably consistent among VITT patients and bind PF4 at the same heparin binding region, allowing the formation of immune complexes without the addition of heparin (FIG. 5). The clustering of the VITT IgG-anti PF4 immune complexes activate the platelets, and potentially other cells through the Fc receptors. The identification of the binding region of VITT IgG on PF4 also offers the potential to develop a highly specific diagnostic test for VITT patients. Although the trigger for this rare vaccine side effect remains elusive, given the highly restricted epitope of the anti-PF4 antibodies and the rarity of this occurrence, this data suggests that VITT could be caused by an existing autoimmune B-cell population against PF4, prior to vaccination.

### Example 2. Materials and Methods

Study Participants

Participants included patients diagnosed with VITT (n=5), patients diagnosed with HIT (n=10), and healthy volunteers (n=10). VITT diagnosis was based on 4 criteria: recent AstraZeneca vaccination, positive for anti-PF4 IgG antibodies, positive in the PF4-enhanced SRA, and no prior exposure to heparin. HIT diagnosis was confirmed using the 4Ts score where all HIT patients had a clinical score of ³4, patients had a positive commercially available PF4 enhanced heparin-dependent IgG/A/M-specific enzyme immunoassay [EIA, Immucor, WI, USA, optical density (OD)≥0.45], and a positive serotonin-release assay (SRA, ≥20% 14C-serotonin release)²⁵. This study was approved by the Hamilton Integrated Research Ethics Board (HiREB) and informed written consent was obtained from all participants.

Platelet Activation Assays

Platelet activation assays were performed in the presence of heparin using the serotonin-release assay (SRA), and including a modification in which increasing doses of PF4 were added, rather than heparin (PF4-SRA).^{25,26} Some assays were performed with high concentrations of unfractionated heparin (100 IU/mL), or with Fc receptor-blocking monoclonal antibody (IV.3).

Epitope Mapping of Antibody-Binding to PF4 from VITT and HIT Patients Using Alanine Scanning Mutagenesis

The full-length DNA coding sequence of human PF4²⁸ was cloned into the pET22b expression vector using restriction sites NdeI and HindIII (GenScript, Piscataway, NJ, USA). The PF4 mutants were expressed and purified as previously described^{7,29}. Briefly, PF4 mutants were designed where non-alanine amino acids in wild-type PF4 were mutated to alanine and the alanine amino acids in wild-type PF4 were mutated to valine. PF4 mutants were introduced into Escherichia coli ArcticExpress (DE3) cells (Agilent Technologies, Santa Clara, CA, USA). Overexpression of PF4 mutant cultures were grown at 37° C. to mid-exponential phase before induction with 0.5 mM isopropyl β-D-1-thiogalactopyranoside (IPTG) and grown at 37° C. for 3 hours. E. coli cells for each wild-type PF4 or PF4 mutant were lysed by sonication in 20 mM sodium phosphate, pH 7.2, 400 mM sodium chloride, 1.4 mM β-mercaptoethanol, 5% (v/v) glycerol, 1% (v/v) Triton X-100 (Thermo Fisher Scientific, Waltham, MA, USA), and 0.5% (w/v) sodium deoxycholate (Sigma-Aldrich, St. Louis, MO, USA) with 2 mM MgCl2, 10 μg/mL DNasel (Sigma-Aldrich, St. Louis, MO, USA) and EDTA-free protease inhibitor cocktail (Roche, Basel, Switzerland). The supernatant was then cleared by centrifugation at 40,000×g for 40 minutes at 4° C. and applied onto a HiTrap Q HP column (Cytiva Life Sciences, Marlborough, MAM, USA) equilibrated with 20 mM sodium phosphate, pH 7.2, 400 mM sodium chloride, 1.4 mM β-mercaptoethanol, and 5% (v/v) glycerol. The flow-through of the Q HP column was then stored at 4° C., overnight. The following day, the serum was diluted 2-fold to yield a sodium chloride concentration of 200 mM with 20 mM sodium phosphate, pH 7.2, 1.4 mM β-mercaptoethanol, and 5% (v/v) glycerol, syringe-filtered with a 0.2 μM filter (Acrodisc, Pall) and loaded onto a HiTrap Heparin HP column (Cytiva Life Sciences, Marlborough, MAM, USA). Contaminants were eluted with 0.5 M sodium chloride and PF4 was eluted with a linear gradient from 0.5 to 2 M sodium chloride. Fractions containing pure wild-type or PF4 mutants were pooled, concentrated and buffer-exchanged to phosphate buffered saline (PBS) and 1.5 M sodium chloride. The concentration of PF4 was determined using a bicinchoninic acid (BCA) assay (Thermo Fisher Scientific, Waltham, MA, USA). Protein expression and purity was assessed for each PF4 mutant using 4-18% sodium dodecyl sulfate polyacrylamide gel electrophoresis (SDS-PAGE).

Measuring the effect of the 70 amino acids on the binding of anti-PF4/heparin antibodies in patient sera was analyzed similar to what has been previously described⁷. The binding of anti-PF4/heparin antibodies to wild-type PF4 and PF4 mutants was measured using a modified PF4/heparin IgG-specific EIA,^{7,16,30}. 384-well NUNC Maxisorp plates (Thermo Fisher Scientific, Waltham, MA, USA) were coated with 10 μg/mL streptavidin and 1 U/mL biotinylated-heparin and blocked with phosphate buffered saline (PBS) supplemented with 3% bovine serum albumin (BSA) for 2 hours at ambient temperature. Wild-type PF4 or PF4 mutants at 5 μg/mL was then added and incubated for 1 hour at ambient temperature. Diluted patient sera ( 1/50 prepared in 1% BSA in PBS) in technical duplicates were added to the plates and incubated for 1 hour at room temperature. After washing, alkaline-phosphatase conjugated goat anti-human IgG (g-chain-specific, Jackson ImmunoResearch Laboratories, Inc, Westgrove, PA, USA) was added at a 1:3,000 dilution and incubated for 1 hour at ambient temperature. Addition of 1 mg/mL p-nitrophenylphosphate (PNPP, Sigma-Aldrich, St. Louis, MO, USA) substrate dissolved in 1 M diethanolamine buffer (pH 9.6) was added for detection. The optical density (OD) was read at 405 nm and 490 nm (as a reference) measured using a BioTek 800TS microplate reader (BioTek, Winooski, VT, USA) to assess binding of antibodies to wild-type PF4 and PF4 mutants. Results were reported as a percentage of binding relative to wild-type PF4 binding.

Binding kinetics of VITT and HIT antibodies using biolayer interferometry (BLI)

Wild-type PF4 was labelled with biotin as previously described¹⁷. Briefly, wild-type PF4 and PF4 mutants were incubated with 5× the volume of Heparin Sepharose 6 Fast Flow affinity chromatography media (Cytiva Life Sciences, Marlborough, MAM, USA) for 1 hour with shaking at ambient temperature. EZ-Link Sulfo-NHS-LC-Biotin (Thermo Fisher Scientific, Waltham, MA, USA) was added to the PF4 and Heparin Sepharose mixture in 20 molar excess and allowed to react for 1 hour with shaking at ambient temperature. The biotinylated wild-type PF4 or PF4 mutants were eluted from the Heparin Sepharose using PBS and 2 M sodium chloride. Absorbance at 280 nm was measured using a spectrophotometer (Eppendorf AG, Hamburg, Germany) and used to calculate the concentration. Biotinylation of PF4 was checked using a streptavidin-coated anti-PF4/heparin EIA. BLI experiments were performed using the Octet-QK Red 96 (ForteBio, Menlo Park, CA, USA). Sera or buffer were dispensed into 96-well black flat-bottom microtiter plates (Greiner Bio-one, Kremsmunster, Austria) at a volume of 200 μL per well with an operating temperature maintained at 30° C. Streptavidin-coated biosensor tips (ForteBio, Menlo Park, CA, USA) were hydrated with PBS supplemented with 1% BSA (Sigma-Aldrich, St. Louis, MO, USA) to establish a baseline prior to antigen immobilization. Biotinylated recombinant PF4 (final concentration 7.5 μg/mL in PBS with 1% BSA) alone or complexed with 0.125 U/mL unfractionated heparin (LEO Pharma, Ballerup, Denmark) were then immobilized on the biosensor tips for 20 minutes at 1,000 rpm followed by an antigen dissociation step in PBS with 1% BSA for 30 minutes at 1,000 rpm. Antigen-coated sensors were then reacted with patient sera at a 1/32 dilution in duplicate in PBS with 1% BSA for 13 minutes at 1,000 rpm followed by a dissociation step for 56 minutes at 1,000 rpm. Thus, each patient serum was tested with biotinylated-PF4 then with biotinylated-PF4/heparin as antigens. Data were analyzed using Octet® User Software version 3.1 using the 2:1 heterogenous ligand binding model. Reference values from control wells were subtracted and all results were aligned to measured baseline. The binding profile response of each serum was expressed as a nm shift (the wavelength/spectral shift in nanometers), representing the difference between the start and end of the association step.

Data Acquisition and Statistical Analysis

Differences between data were tested for statistical significance using the paired or unpaired t-test and the Mann-Whitney test. P-values are reported as 2-tailed and a p-value of <0.05 was considered to be statistically significant. All statistical analyses were conducted using GraphPad Prism (version 9.1.0, GraphPad Software, San Diego, USA).

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

TABLE 1
Sequences of the disclosure.

| SEQ ID NO: | Amino Acid Sequence |
|---|---|
| 1 | EAEEDGDLQCLCVKTTSQVRPRHIT |
| | SLEVIKAGPHCPTAQLIATLKNGRK |
| | ICLDLQAPLYKKIIKKLLES |

### FULL CITATIONS FOR DOCUMENTS REFERRED TO IN THE APPLICATION

1 Greinacher, A. et al. Thrombotic Thrombocytopenia after ChAdOx1 nCov-19 Vaccination. N Engl J Med, (2021).
2 Schultz, N. H. et al. Thrombosis and Thrombocytopenia after ChAdOx1 nCoV-19 Vaccination. N Engl J Med, (2021).
3 Scully, M. et al. Pathologic Antibodies to Platelet Factor 4 after ChAdOx1 nCoV-19 Vaccination. N Engl J Med, (2021).
4 Kelton, J. G. et al. Heparin-induced thrombocytopenia: laboratory studies. Blood 72, 925-930 (1988).
5 Kelton, J. G. & Warkentin, T. E. Heparin-induced thrombocytopenia: a historical perspective. Blood 112, 2607-2616, (2008).
6 Warkentin, T. E. & Kelton, J. G. Temporal aspects of heparin-induced thrombocytopenia. N Engl J Med 344, 1286-1292, (2001).
7 Huynh, A. et al. Characterization of platelet factor 4 amino acids that bind pathogenic antibodies in heparin-induced thrombocytopenia. J Thromb Haemost 17, 389-399, (2019).
8 Warkentin, T. E., Makris, M., Jay, R. M. & Kelton, J. G. A spontaneous prothrombotic disorder resembling heparin-induced thrombocytopenia. Am J Med 121, 632-636, (2008).
9 Hwang, S. R. et al. Cerebral venous sinus thrombosis associated with spontaneous heparin-induced thrombocytopenia syndrome after total knee arthroplasty. Platelets, 1-5, (2020).
10 Amiral, J. et al. Antibodies to macromolecular platelet factor 4-heparin complexes in heparin-induced thrombocytopenia: a study of 44 cases. Thromb Haemost 73, 21-28 (1995).
11 Ziporen, L. et al. Defining an antigenic epitope on platelet factor 4 associated with heparin-induced thrombocytopenia. Blood 92, 3250-3259 (1998).
12 Li, Z. Q. et al. Defining a second epitope for heparin-induced thrombocytopenia/thrombosis antibodies using KKO, a murine HIT-like monoclonal antibody. Blood 99, 1230-1236, (2002).
13 Lee, D. H., Warkentin, T. E., Denomme, G. A., Hayward, C. P. & Kelton, J. G. A diagnostic test for heparin-induced thrombocytopenia: detection of platelet microparticles using flow cytometry. Br J Haematol 95, 724-731, (1996).
14 Rauova, L. et al. Monocyte-bound PF4 in the pathogenesis of heparin-induced thrombocytopenia. Blood 116, 5021-5031, (2010).
15 Linkins, L. A. et al. Combination of 4Ts score and PF4/H-PaGIA for diagnosis and management of heparin-induced thrombocytopenia: prospective cohort study. Blood 126, 597-603, (2015).
16 Horsewood, P., Warkentin, T. E., Hayward, C. P. & Kelton, J. G. The epitope specificity of heparin-induced thrombocytopenia. Br J Haematol 95, 161-167, (1996).
17 Huynh, A. et al. The role of fluid-phase immune complexes in the pathogenesis of heparin-induced thrombocytopenia. Thromb Res 194, 135-141, (2020).
18 Vayne, C. et al. Characterization of New Monoclonal PF4-Specific Antibodies as Useful Tools for Studies on Typical and Autoimmune Heparin-Induced Thrombocytopenia. Thromb Haemost 121, 322-331, (2021).
19 Nguyen, T. H. & Greinacher, A. Distinct Binding Characteristics of Pathogenic Anti-Platelet Factor-4/Polyanion Antibodies to Antigens Coated on Different Substrates: A Perspective on Clinical Application. ACS Nano 12, 12030-12041, (2018).
20 Warkentin, T. E. et al. Combination of two complementary automated rapid assays for diagnosis of heparin-induced thrombocytopenia (HIT). J Thromb Haemost 18, 1435-1446, (2020).
21 Warkentin, T. E., Sheppard, J. I., Linkins, L. A., Arnold, D. M. & Nazy, I. High sensitivity and specificity of an automated IgG-specific chemiluminescence immunoassay for diagnosis of HIT. Blood 132, 1345-1349, (2018).
22 Dennison, S. M. et al. Qualified Biolayer Interferometry Avidity Measurements Distinguish the Heterogeneity of Antibody Interactions with Plasmodium falciparum Circumsporozoite Protein Antigens. J Immunol 201, 1315-1326, (2018).
23 Ylera, F., Harth, S., Waldherr, D., Frisch, C. & Knappik, A. Off-rate screening for selection of high-affinity anti-drug antibodies. Anal Biochem 441, 208-213, (2013).
24 Sachais, B. S. et al. Dynamic antibody-binding properties in the pathogenesis of HIT. Blood 120, 1137-1142, (2012).
25 Sheridan, D., Carter, C. & Kelton, J. G. A diagnostic test for heparin-induced thrombocytopenia. Blood 67, 27-30 (1986).
26 Nazy, I. et al. Distinguishing between anti-platelet factor 4/heparin antibodies that can and cannot cause heparin-induced thrombocytopenia. J Thromb Haemost 13, 1900-1907, (2015).
27 Rubino, J. G. et al. A comparative study of platelet factor 4-enhanced platelet activation assays for the diagnosis of heparin-induced thrombocytopenia. J Thromb Haemost 19, 1096-1102, (2021).
28 Deuel, T. F. K., P. S.; Farmer, M.; Heinrikson, R. L. Amino acid sequence of human platelet factor 4. Proc Natl Acad Sci USA 74, 2256-2258 (1977).
29 Huynh, A. et al. Development of a high-yield expression and purification system for platelet factor 4. Platelets 29, 249-256, (2018).
30 Stearns, N. A., Prigent-Richard, S., Letourneur, D. & Castellot, J. J., Jr. Synthesis and characterization of highly sensitive heparin probes for detection of heparin-binding proteins. Anal Biochem 247, 348-356, (1997).
31 Mayo K H, Ilyina E, Roongta V, et al. Heparin binding to platelet factor-4. An NMR and site-directed mutagenesis study: arginine residues are crucial for binding. Biochem J. 1995;312 (Pt 2):357-365.
32 Huynh A, Kelton J G, Arnold D M, Daka M, Nazy I. Antibody epitopes in vaccine-induced immune thrombotic thrombocytopaenia. Nature. 2021;596(7873):565-569.
33 Warkentin T E, Sheppard J I, Moore J C, Sigouin C S, Kelton J G. Quantitative interpretation of optical density measurements using PF4-dependent enzyme-immunoassays. J Thromb Haemost. 2008;6(8):1304-1312.
34. Sheridan D, Carter C, Kelton J G. A diagnostic test for heparin-induced thrombocytopenia. Blood. 1986;67(1):27-30.

What is claimed:

1. A mutant Platelet Factor 4 (PF4) protein comprising a mutation to at least one amino acid selected from positions R20, H23, T25, E28, K46, R49, K50, K62, K65, or K66 of the amino acid sequence of SEQ ID NO:1.

2. The mutant PF4 protein of claim 1, wherein the mutation comprises substitution of one or more mutations selected from R20A, H23A, T25A, E28A, K46A, R49A, K50A, K62A, K65A, or K66A to the amino acid sequence of SEQ ID NO:1.

3. The mutant PF4 protein of claim 1, wherein the mutant PF4 protein:
(a) selectively binds a heparin-induced thrombocytopenia (HIT) antibody compared to a vaccine-induced immune thrombotic thrombocytopenia (VITT) antibody (PF4-H⁺V⁻);
(b) selectively binds the HIT antibody compared to a non-HIT antibody (PF4-H⁺NH⁻); and/or
(c) selectively binds the HIT antibody compared to both the VITT antibody and the non-HIT antibody (PF4-H⁺V⁻NH⁻).

4. The mutant PF4 protein of claim 3, wherein:
i) the PF4-H⁺V⁻ comprises the substitution R20A, H23A, T25A, E28A, K46A, R49A, K50A, K62A, K65A, or K66A to the amino acid sequence of SEQ ID NO:1;
ii) the PF4-H⁺NH⁻ comprises the substitution H23A, T25A, R49A, K65A, or K66A to the amino acid sequence of SEQ ID NO:1; and/or
iii) the PF4-H⁺V⁻NH⁻ comprises the substitution H23A, T25A R49A, K65A, or K66A to the amino acid sequence of SEQ ID NO:1.

5. The mutant PF4 protein of claim 3, wherein the PF4-H⁺V⁻ comprises the substitution H23A, E28A, K50A, K62A, or K66A to the amino acid sequence of SEQ ID NO:1.

6. The mutant PF4 protein of claim 3, wherein the PF4-H⁺V⁻ comprises the substitution H23A, E28A, K50A, or K66A to the amino acid sequence of SEQ ID NO:1.

7. The mutant PF4 protein of claim 3, wherein the PF4-H⁺NH⁻ comprises the substitution H23A, T25A, or K65A to the amino acid sequence of SEQ ID NO:1.

8. A method of differentiating between VITT, HIT, and non-HIT in a sample from a patient suspected of having VITT, HIT or non-HIT comprising:
a) contacting the sample with at least one of the mutant PF4 protein according to claim 1 and measuring binding of the at least one mutant PF4 protein with the sample;
b) contacting the sample with the PF4 protein comprising the amino acid sequence of SEQ ID NO:1 and measuring the binding of the PF4 protein with the sample;
c) determining a ratio of the binding in a) to the binding in b); and
d) determining the level of similarity of the ratio in c) to one or more control profiles, wherein (i) a high level of similarity to a HIT specific control profile or a low level of similarity to a VITT specific control profile and/or a non-HIT specific control profile is indicative of HIT; (ii) a high level of similarity to the VITT specific control profile or a low level of similarity to the HIT specific control profile and/or the non-HIT specific control profile is indicative of VITT; and/or (iii) a high level of similarity to the non-HIT specific control profile or a low level of similarity to the HIT specific control profile and/or the VITT specific control profile is indicative of non-HIT.

9. The method of claim 8, first comprising the step of immobilizing the mutant PF4 protein in a) and/or the PF4 protein in b) on a support, optionally a biosensor tip or microplate. The method of claim 8, wherein the mutant PF4 protein in a) and/or the PF4 protein in b) is biotinylated.

11. The method of claim 9, wherein the mutant PF4 protein in a) and/or the PF4 protein in b) is immobilized alone or complexed with unfractionated heparin.

12. The method of claim 8, wherein the method comprises enzyme-linked immunosorbent assay (ELISA), enzyme immunoassay (EIA), or bio-layer interferometry (BLI).

13. The method of claim 8, wherein the measuring in step a) and/or b) comprises measuring the detection of a secondary antibody tagged with a detectable label against the HIT antibody, the VITT antibody, and/or the non-HIT antibody bound to the mutant PF4 protein or the PF4 protein comprising the amino acid sequence of SEQ ID NO:1.

14. The method of claim 8, wherein the measuring in step a) and/or step b) comprises measuring a colorimetric shift in optical interference pattern induced by binding of the HIT antibody, the VITT antibody, and/or the non-HIT antibody to the mutant PF4 protein or the PF4 protein comprising the amino acid sequence of SEQ ID NO:1.

15. A method of differentiating between VITT and HIT in a sample from a patient suspected of having VITT or HIT comprising:
I) combining the sample with platelets labeled with serotonin;
II) incubating the combined sample and platelets with no unfractionated heparin and at least two concentrations of the unfractionated heparin, and measuring serotonin release to the no unfractionated heparin and to the at least two concentrations of the unfractionated heparin;
III) incubating the combined sample and platelets with no mutant PF4 protein and at least two concentrations of the mutant PF4 protein according to claim 1, and measuring the serotonin release to the no mutant PF4 protein and to the at least two concentrations of the mutant PF4 protein;
IV) measuring a dose response of the sample in II) and in III); and
V) determining the level of similarity of the dose response in IV) to one or more control profiles, wherein (i) a high level of similarity to a HIT specific control profile or a low level of similarity to a VITT specific control profile is indicative of HIT; and/or (ii) a high level of similarity to the VITT specific control profile or a low level of similarity to the HIT specific control profile is indicative of VITT.

16. The method of claim 15, wherein the platelets in I) are preincubated with FcγRIIa-blocking monoclonal antibody (IV.3).

17. The method of claim 15, wherein the HIT specific control profile comprises a non-null dose response to both i) the unfractionated heparin, and ii) the mutant PF4 protein; and/or wherein the VITT specific control profile comprises a null dose response to i) the unfractionated heparin, and the non-null dose response to ii) the mutant PF4 protein.

18. The method of claim 8, wherein the sample is a blood sample, optionally a serum or plasma sample.
